(19) United States
(12) Patent Application Publication
(10) Pub. No.: US 2025/0079002 A1
(43) Pub. Date: Mar. 6, 2025

(54) INTERPRETABLE DOMAIN ADAPTATION FOR OPTIMIZING CROSS-COHORT PREDICTIONS FROM MEDICAL DATA

(71) Applicant: NEC Laboratories Europe GmbH, Heidelberg (DE)

(72) Inventors: Raman Siarheyeu, Heidelberg (DE); Zhao Xu, Heidelberg (DE)

(21) Appl. No.: 18/497,064

(22) Filed: Oct. 30, 2023

Related U.S. Application Data
(60) Provisional application No. 63/534,878, filed on Aug. 28, 2023.

(51) Int. Cl. G16H 50/20; G16H 10/60

(52) U.S. Cl. CPC G16H 50/20; G16H 10/60

(57) ABSTRACT

A computer-implemented, machine learning method for cross-cohort predictions from medical data. Patients of one or more source cohorts are mapped to a feature space of a target cohort based on constraints. Patient distributions of the one or more source cohorts and the target cohort are learned. The patient distributions of the one or more source cohorts are corrected for the target cohort. The method has applications including, but not limited to medical AI, drug development, medical diagnostics/applications and in healthcare, for example, to optimize predictions or support decision making.

### CROSS-REFERENCE TO PRIOR APPLICATION

Priority is claimed to U.S. Provisional Application Ser. No. 63/534,878 filed on Aug. 28, 2023, the entire contents of which is hereby incorporated by reference herein.

### FIELD

The present invention relates to Artificial Intelligence (AI) and machine learning (ML), and in particular to medical AI and a method, system, data structure, computer program product and computer-readable medium for interpretable domain adaptation for cross-cohort predictions, such as disease predictions, from medical data, such as microbiome data.

### SUMMARY

In an embodiment, the present invention provides a computer-implemented, machine learning method for cross-cohort predictions from medical data. Patients of one or more source cohorts are mapped to a feature space of a target cohort based on constraints. Patient distributions of the one or more source cohorts and the target cohort are learned. The patient distributions of the one or more source cohorts are corrected for the target cohort. The method has applications including, but not limited to medical AI, drug development, medical diagnostics/applications and in healthcare, for example, to optimize predictions or support decision making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a system and method according to an embodiment of the present invention;

FIG. 2 schematically illustrates a process of making predictions for a new patient from a target cohort according to an embodiment of the present invention; and

FIG. 3 illustrates is a block diagram of an exemplary processing system, which can be configured to perform any and all operations disclosed herein.

### DETAILED DESCRIPTION

Embodiments of the present invention provide improvements to machine learning disease diagnostics using microbiome information. In doing so, embodiments of the present invention provide to overcome the technical problem of the highly heterogeneous nature of microbiome data, which makes it challenging to make accurate or reliable predictions to begin with, much less to interpret estimated performance of ML models. The ML models can be overoptimistic in single-cohort settings or significantly degrade in performance when analyzing patients from multiple cohorts. Embodiments of the present invention enable microbiome data integration to build a disease model across multi-cohorts and make predictions with explanations. This not only increases accuracy and performance of AI systems and ML models, but also provide the end-user an insight about the reliability of the model predictions and increases the trust in the quality of predictions or diagnostics.

The microbiome has emerged as a promising indicator for human diseases. Recent studies revealed direct and indirect associations between microorganisms and numerous conditions, such as irritable bowel syndrome (IBD), chronic kidney diseases, pancreatic cancer, inflammatory bowel disease, and colorectal cancer (CRC). Shifting microbial compositions have been associated with the onset and progression of diabetes, obesity, tuberculosis, and autism spectrum disorder. Moreover, several studies have highlighted the relationship between gut microbiota composition and the effectiveness of cancer chemotherapies and immunotherapies. Those findings underscore the potential benefits and utility of microbiome as a diagnostic tool, enabling early detection and personalized approaches to disease management (see Huang, K., Wu, L. and Yang, Y., “Gut microbiota: An emerging biological diagnostic and treatment approach for gastrointestinal diseases,” JGH Open, 5: 973-975 (2021); and Chauhan, N. S., Mukerji, M. and Gupta, S., “Editorial: Role of microbiome in diseases diagnostics and therapeutics,” Frontiers in Cellular and Infection Microbiology 12:1025837 (2022), each of which is hereby incorporated by reference herein).

The increasing number of metagenome sequencing experiments and the complexity of microbiome data have led to the widespread adoption of machine learning approaches in the microbiome research. Different methods were developed to detect the co-occurrence patterns in microbial communities and predict an environmental or host phenotypes. However, translating machine learning microbiome diagnosis into clinical practice presents several technical obstacles. Achieving high accuracy and low uncertainty of the predictions requires large amount of high-quality and correctly labeled data for training.

The diverse nature of the human microbiome complicates data acquisition and model training. Microbiome data is significantly affected by the presence of wide inter-individual heterogeneity in microbiota composition, cohort-dependent effects and technical biases. These factors can hinder the generalization of machine learning models raising questions about the true performance of disease-predictive models in microbiome studies.

For instance, geographic location of the patients is known to have a significant impact on the variations in their microbiome and cause cohort-dependent effects. While building machine learning models based on less heterogeneous data from local cohorts processed under a standardized regime is a potential approach for microbiome-based predictions, such cohorts often contain only a limited number of samples. This technical limitation necessitates the exploration of alternative strategies, such as adopting of well-validated models for a same disease trained on other studies or training the whole model on the data aggregated over multiple cohorts (see He, Y., Wu, W., Zheng. H. M., et al. “Regional variation limits applications of healthy gut microbiome reference ranges and disease models.” Nature Medicine 24, 1532-1535 (2018), which is hereby incorporated by reference herein).

However, models transferred between studies demonstrate less accuracy in cross-study analysis than models tested by within-study cross-validation. To illustrate the impact of cohort effects on model performance, Pasolli, E., Truong, D. T., Malik, F., Waldron, L. and Segata, N., “Machine Learning Meta-analysis of Large Metagenomic Datasets: Tools and Biological Insights,” PLOS Comput Biol 12(7): e1004977 (2016), hereinafter “Pasolli et al.”, which is hereby incorporated by reference herein, investigated datasets from populations with distinct characteristics. This study demonstrated that cohort effects influence model results, and the prediction on independent cohorts leads to significantly reduced accuracy. As suggested in Li. M., Liu, J., Zhu, J., Wang. H., Sun, C., Gao, N. L., Zhao, X. and Chen, W. et al., “Performance of Gut Microbiome as an Independent Diagnostic Tool for 20 Diseases: Cross-Cohort Validation of Machine-Learning Classifiers,” Gut Microbes, 15:1 (2023), which is hereby incorporated by reference herein, pooling of training cohorts could improve predictive performances in independent cohorts for most diseases. However, even when multiple cohorts were combined as the training set, there were still very low external AUCs (area under the (receiver operating characteristic) curve) observed. For some diseases, like CRC and Crohn Disease, there were not always observed increased external AUCs with the increasing numbers of cohorts.

Interestingly, multiple authors report that addition of healthy (control) samples from other studies to the training cohort may improve disease prediction. As reported by Pasolli et al., cross-study validation of type 2 diabetes classification was improved by adding gut microbiome samples from the healthy subjects of four other datasets, i.e., cirrhosis, CRC, human microbiome project, and IBD, to the training data. The leveraging schema in Song. K. and Zhou, Y. H., “Leveraging Scheme for Cross-Study Microbiome Machine Learning Prediction and Feature Evaluations,” Bioengineering 10(2):231, Basel (2023), hereinafter “Song et al.”, which is hereby incorporated by reference herein, also suggests construction of the predictive models by combining a portion of external data (target data) into a larger and independent data set (source data) for prediction and evaluation of the remaining portion of the external (target) data, claiming that using at least 25% of the target samples in the source data resulted in improved model performance.

The technical challenges highlighted above raise the question concerning the generalization capabilities of machine learning models in the context of microbiome-based disease diagnostics and the applicability of the model predictions in cross-cohort settings. Consequently, identifying the strategies of utilizing data for training machine learning models across multiple cohorts, as well as providing well-interpretable insights about the reliability of the model predictions, would allow for facilitating the practical implementation of these models in clinical practice.

Embodiments of the present invention provide a method and a system that allows interpretable sample integration to build a disease model with multi-cohort microbiome data for accurate, reliable and explainable predictions. This also gives an insight to the end-user about the reasoning behind the model predictions in cross-cohort settings, thereby enhancing the trust in the predictions and increasing the quality of the final decision making.

Embodiments of the present invention provide interpretable domain adaptation and explainability in ML-based disease prediction from multi-cohort microbiome data. The method learns to correct the patient distributions of different cohorts, such that an accurate disease model of the target cohort can be learned from the corrected source cohorts.

In a first aspect, the present invention provides a computer-implemented, machine learning method for cross-cohort predictions from medical data. Patients of one or more source cohorts are mapped to a feature space of a target cohort based on constraints. Patient distributions of the one or more source cohorts and the target cohort are learned. The patient distributions of the one or more source cohorts are corrected for the target cohort.

In a second aspect, the present invention provides the method according to the first aspect, wherein the constraints include a proximity of samples in the one or more source cohorts, and the proximity about disease conditions.

In a third aspect, the present invention provides the method according to the first or second aspect, wherein mapping the patients of the or more source cohorts to the feature space of the target cohort includes training an encoder model to embed the patients of the one or more source cohorts to the feature space of the target cohort, and wherein the constraints are utilized in a loss function as separate regularization terms while training the encoder model.

In a fourth aspect, the present invention provides the method according to any of the first to third aspects, wherein learning the patient distributions of the one or more source cohorts includes training a variational autoencoder to determine specific distributions for the target cohort and the one or more source cohorts.

In a fifth aspect, the present invention provides the method according to any of the first to fourth aspects, wherein correcting the patient distributions of the one or more source cohorts for the target cohort is based on generative deep learning and determining an alignment rate.

In a sixth aspect, the present invention provides the method according to any of the first to fifth aspects, further comprising learning a disease model of the target cohort using the one or more source cohorts that have corrected the patient distributions.

In a seventh aspect, the present invention provides the method according to any of the first to sixth aspects, further comprising predicting disease for a new patient based on the learned disease model.

In an eighth aspect, the present invention provides the method according to any of the first to seventh aspects, wherein the disease model revises loss by weighting the patients of the one or more source cohorts with an associated alignment rate.

In a ninth aspect, the present invention provides the method according to any of the first to eighth aspects, wherein the learned disease model generates one or more explanations for predicting the disease for the new patient.

In a tenth aspect, the present invention provides the method according to any of the first to ninth aspects, wherein the one or more explanations are based on genuine patients and/or synthetic patients used to obtain the disease prediction.

In an eleventh aspect, the present invention provides the method according to any of the first to tenth aspects, wherein learning the patient distributions of the one or more source cohorts includes using the trained variational autoencoder or conditional variational autoencoder to learn the patient distributions separately, including pₜ(z|x) and pₜ(x|z) for the target cohort and pₛ(z|x) and pₛ(x|z) for each cohort of the one or more source cohorts.

In a twelfth aspect, the present invention provides the method according to any of the first to eleventh aspects, wherein the medical data includes microbiome data, wherein the alignment rate is a coefficient δ that specifies similarity of a patient i of a source cohort s of the one or more source cohorts is to the target cohort t, wherein correcting the patient distributions of the one or more source cohorts for the target cohort includes analyzing the patients of the source cohort s and the target cohort t by:

$${δ}_{t} ( {x}_{i}^{( s )} ) = \frac{{p}_{t} ( {x}_{i}^{( s )} )}{{p}_{s} ( {x}_{i}^{( s )} )} ,$$

wherein xᵢ^{(s)} denotes the microbiome data of the patient i of the source cohort s, wherein probability pₜ(xᵢ^{(s)}) is determined by:

$${p}_{t} ( {x}_{i}^{( s )} ) = \int {p}_{t} ( {z}_{i} ) {p}_{t} ( {x}_{i}^{( s )} \vert {z}_{i} ) {dz}_{i} \approx {\sum }_{j = 1}^{N} {p}_{t} ( {z}_{j} ) {p}_{t} ( {x}_{i}^{( s )} \vert {z}_{j} ) ,$$

wherein the term pₜ(xᵢ^{(s)}|zⱼ) is determined using the learned patient distributions, wherein pₜ(z) is determined by randomly drawing K patients from the target cohort and determining pₜ(z|xₖ^{(t)}) for each patient of the randomly drawn K patients, and wherein a mean distribution of the K patients is an empirical estimation of pₜ(z|x).

In a thirteenth aspect, the present invention provides the method according to any of the first to twelfth aspects, wherein the medical data includes microbiome data, wherein learning the disease model includes using a neural network that generates disease labels as outputs and the microbiome data xᵢ^{(s)} of the corrected patient distributions of a corresponding source cohort of the one or more source cohorts and the microbiome data xⱼ^{(t)} of the corrected patient distributions of the target cohort as inputs, wherein parameters of the neural network are represented by Φ, wherein revising the loss includes:

$$Loss = {\sum }_{j = 1}^{M} - log p ( {y}_{j}^{( t )} \vert {x}_{j}^{( t )} , Φ ) + {\sum }_{s = 1}^{S} {\sum }_{i = 1}^{N} - log {δ}_{t} ( {x}_{i}^{( s )} ) p ( {y}_{i}^{( s )} \vert {x}_{i}^{( s )} , Φ ) ,$$

wherein p(yⱼ^{(t)}|xⱼ^{(t)}, Φ) represents predictions with data from the target cohort, and wherein p(yᵢ^{(s)}|xᵢ^{(s)}, Φ) represents the predictions with the data from the one or more source cohorts.

In a fourteenth aspect, the present invention provides a computer system for cross-cohort predictions from medical data comprising one or more processors which, alone or in combination, are configured to perform a machine learning method for cross-cohort predictions from medical data according to any of the first to thirteenth aspects.

In a fifteenth aspect, the present invention provides a tangible, non-transitory computer-readable medium for cross-cohort predictions from medical data containing instructions which, upon being executed by one or more hardware processors, provide for execution of a machine learning method according to any of the first to thirteenth aspects.

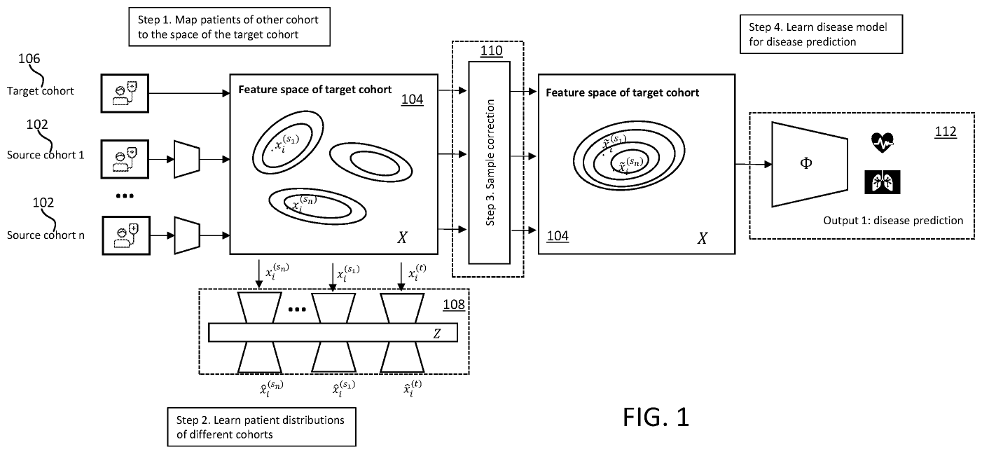

FIG. 1 provides a high-level overview of the method and system according to an embodiment of the present invention.

### Step 2.1—Map Patients of Other Cohorts to the Space of the Target Cohort:

Microbiome data of patients in different study cohorts or hospitals are often obtained with related but different protocols, sequencing platforms, and analysis tools, which cause discrepancies. This means it is not possible to directly use patients' microbiome data of different cohorts. Embodiments of the present invention overcome this technical problem by mapping them into the same space. To overcome this technical problem the present invention can compute similarities by using any type of microbiome features, for example, species abundances or gene marker presence. To this end, an embodiment of the present invention introduces an encoder to embed the patients of other cohorts (denoted as source domains or source cohorts 102) to the feature space 104 of the target hospital (denoted as target domain or target cohort 106). Beyond a standard encoder, the mapping satisfies the following constraints:
Keeping proximity of the patients in the source domain. Specifically, if two patients are close to each other in the source domain, then they are still similar after mapping.
Keeping proximity between the patients of the source domain and those of the target domain. Specifically, if two patients from different cohorts 102 have the same disease, then they should possess similar features after mapping, compared with two patients of different disease conditions.

Those constraints are satisfied, for example, by taking them into account in the loss function as separate regularization terms while training the encoder model. In embodiments, the encoder can be trained for satisfying the first constraint by randomly selecting two pairs of patients. If the distance of the first pair of patients is lower than that of the second pair, i.e., dist(patient_i, patient_j)>dist (patient_i′, patient_j′), then after mapping the relation remains the same. For the second constraint and for a patient_i in a source domain (cohort), the encoder randomly selects a patient_i′ of the same disease and a patient_j′ of a different disease from a target cohort. After mapping, the distance of patient_i and patient_i′ should be larger than that of patient_i and patient_j′, i.e., dist(patient_i, patient_j′)>dist(patient_i, patient_j′).

### Step 2.2—Learn Patient Distributions of Different Cohorts

The patients of different study cohorts or hospitals often live in different locations with different lifestyles and environments. Even with mapping all patients into the space of the target cohort 106, the distributions of the data about the same disease diverge to some extent. Embodiments of the present invention overcome this technical problem by providing to learn distinguishing domain-specific distributions of patients. To do so, an embodiment of the present invention employs a variational autoencoder (VAE) (see Kingma, D. P. and Welling, M., “Auto-Encoding Variational Bayes,” ICLR (2014), which is hereby incorporated by reference herein) or a conditional VAE (see Sohn, K., Lee, H. and Yan, X., “Learning Structured Output Representation using Deep Conditional Generative Models,” NIPS (2015), which is hereby incorporated by reference herein) to learn the distributions separately, including: pₜ(z|x) and pₜ(x|z) for the target cohort 106 and pₛ(z|x) and pₛ(x|z) for each source cohort 102. The VAE is trained at this step preferably independently of the encoder model of Step 2.1. In embodiments, a patient distribution module 108 may be used to train and implement the VAE and/or the conditional VAE.

### Step 2.3—Sample Correction:

The patients of the source cohorts 102 are distributed differently from the target cohort 106, and therefore it would not be effective to simply put all the data together. Embodiments of the present invention overcome this technical problem by providing to correct the samples from different cohorts. In particular, an embodiment of the present invention defines a coefficient, alignment rate (denoted as δ), to specify how similar a patient i of a source cohort (102) s is with respect to the target cohort (106) t by considering the entirety of the patients of the two cohorts:

$${δ}_{t} ( {x}_{i}^{( s )} ) = \frac{{p}_{t} ( {x}_{i}^{( s )} )}{{p}_{s} ( {x}_{i}^{( s )} )} ,$$

where xᵢ^{(s)} denotes microbiome data of the patient i of the source cohort (102) s. The probability pₜ(xᵢ^{(s)}) is computed as follows:

$${p}_{t} ( {x}_{i}^{( s )} ) = \int {p}_{t} ( {z}_{i} ) {p}_{t} ( {x}_{i}^{( s )} \vert {z}_{i} ) {dz}_{i} \approx {\sum }_{j = 1}^{N} {p}_{t} ( {z}_{j} ) {p}_{t} ( {x}_{i}^{( s )} \vert {z}_{j} ) .$$

The term pₜ(xᵢ^{(s)}|zⱼ) can be computed directly with the output of Step 2.2. To compute pₜ(z), K patients are randomly drawn from the target cohort 106, thereby obtaining pₜ(z|xₖ^{(t)}) for each. Then the mean distribution of the K patients is an empirical estimation of pₜ(z|x). Since the sum of independent Gaussian distributions is still Gaussian, the following is obtained:

$${p}_{t} ( z ) \approx \frac{1}{K} {\sum }_{k = 1}^{K} {p}_{t} ( z \vert {x}_{k}^{( t )} ) \\ {z}_{j}^{( t )} ∼ {p}_{t} ( z ) and {ω}_{j} = {p}_{t} ( {z}_{j}^{( t )} )$$

Sample correction is a technical problem in many areas (see Cortes, C., Mohri, M., Riley, M. and Rostamizadeh, A., “Sample Selection Bias Correction Theory,” arXiv:0805.2775 (2008), which is hereby incorporated by reference herein). Also, estimation of the data distributions pₜ and pₛ are technically challenging. Embodiments of the present invention overcome these technical problems with a generative deep learning-based method to approximate effectively. In embodiments a sample correction module 110 may be used to implement the sample correction to specify how similar a patient i of a source cohort (102) s is with respect to the target cohort (106) t by considering the entirety of the patients of the two cohorts.

### Step 2.4—Learn the Disease Model of the Target Cohort:

In this step, embodiments of the present invention now learn the disease model of the target cohort 106 with its own patient data and the patient data from other cohorts (source cohorts) 102. The disease model is flexible, and can be any neural network with the disease labels as outputs and the converted patient microbiome data xᵢ^{(s)} of source cohorts 102 and patient microbiome data xⱼ^{(t)} of target cohort 106 as inputs. The parameters of the neural network are denoted as Φ, which will be learned with the patient data. In embodiments, the parameters are attributes of the neural network. Examples may include the weights matrix and bias vectors of the neural network. The sample correction is used to revise the loss: patients from other cohorts 102 will be weighted with their alignment rate (outputs of Step 2.3):

$$Loss = {\sum }_{j = 1}^{M} - log p ( {y}_{j}^{( t )} \vert {x}_{j}^{( t )} , Φ ) + {\sum }_{s = 1}^{S} {\sum }_{i = 1}^{N} - log {δ}_{t} ( {x}_{i}^{( s )} ) p ( {y}_{i}^{( s )} \vert {x}_{i}^{( s )} , Φ ) ,$$

where p(yⱼ^{(t)}|xⱼ^{(t)}, Φ) represents the predictions with only data from the target cohort 106, p(yᵢ^{(s)}|xᵢ^{(s)}, Φ) represents the predictions with data added from the source cohorts 102.

The disease model can be trained with any optimization algorithm, e.g., stochastic gradient descent and implemented by a disease model generation module 112.

### Step 2.5—Predict Disease for a New Patient:

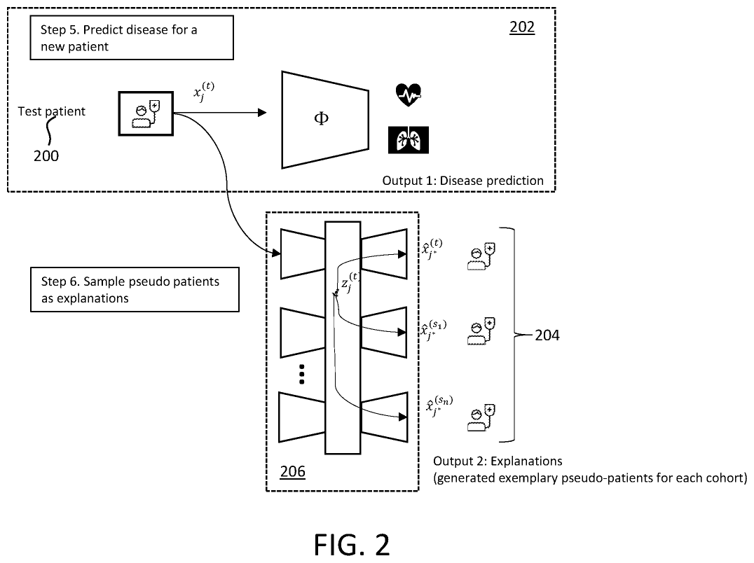

Using the learned disease model, embodiments of the present invention provide to predict a disease for a new patient (test patient 200) of the target cohort. In particular, where the target cohort receives a new patient 200 with microbiome features x_{*}^{(t)} as shown in FIG. 2, the disease model learned in Step 2.4 can be used to predict the new patient's 200 risk level p(y_{*}^{(t)}|x_{*}^{(t)}, Φ) about a disease y_{*}^{(t)}. In embodiments a disease prediction module 202 may be used to determine the risk level using the learned disease model for a new patient 200. It is to be understood that using microbiome features for disease prediction as described herein represents an exemplary, advantageous embodiment, and the present disclosure is not limited to microbiome data or disease prediction. In particular, the methods described herein can be applied to other classifications using medical data, for example for predicting treatments or interventions for patients. Medical data includes, but is not limited to, microbiome data, proteomic data, data derived from laboratory examinations, microarray data, as well as other data sets where cohort biases are present.

### Step 2.6—Generate Explanations of the Predictions:

In addition, embodiments of the present invention can enhance reliability and trust in the AI system by providing human-understandable explanations 204 for the model predictions. For example, embodiments of the present invention provide explanations 204 that identify similar patients from different cohorts for a test patient 200 as shown in FIG. 2. Here, embodiments of the present invention provide the distinguishing advantage of using the learned distribution of the entirety of the patients to get similar patients. The commonly used similarity computation, e.g., Manhattan distance and Cosine similarity, does not work because of high dimensionality of microbiome data (˜2000 dimensional). Embodiments of the present invention provide to overcome this technical problem. In particular, according to an embodiment, the following steps are performed to provide explanations:
The learned pₜ(z|x) and pₜ(x|z) of the target cohort is used to identify the patients in the same cohort as an explanation 204.
The learned alignment rate δₜ(xᵢ^{(s)}) of each source cohort is used to identify the patients in different cohort as an explanation 204.

If there are privacy requirements, embodiments of the present invention provide to generate pseudo-patients with the learned models to maintain privacy. An explanation generation module 206 may be used to generate explanations 204. For a test patient, the system can find similar real patients. As shown in 206, latent vectors may be sampled from the distribution of similar patients with the encoders. Based on the sampled latent vectors, features of pseudo patients can be constructed with decoders. The pseudo patients are similar with the real patients (e.g., following their distributions), but different.

Embodiments of the present invention can be practically applied to a number of use cases in digital medicine and personalized healthcare, for example, for disease prediction, patient stay predictions, personalized treatment design and AI-assisted drug development or vaccine development. The following are exemplary embodiments applied to different exemplary use cases.

In a first exemplary embodiment, the present invention can be applied for interpretable disease prediction with multi-hospital microbiome data. Here, a use case is to provide predictions and explanations of clinical risk for patients based on their microbiome data. Microbiome data has been approved as an effective diagnostic tool, and could be used to enable early detection and personalized approaches to disease management. However, due to the complexity of the microbiome data, it is technically challenging to beneficially integrate the data of multiple hospitals and to learn an accurate disease model for a target hospital. In addition, the predicted clinical risk being explained would provide for transparency of the AI system, especially for the highly risky healthcare area. In this use case, the data source includes patient microbiome data of target hospital and other hospitals. Application of the method according to an embodiment of the present invention provides to learn an explainable disease model for the target hospital by considering all patient microbiome data of different hospitals. As automated decisions or actions (technicity), the prediction, for a new patient, of clinical risk, together with explanations of the prediction can be used to begin treatment or generate effective treatments for the predicted disease. The explanations can be genuine patients identified from different hospitals or synthetic patients generated by the invention (e.g., to maintain privacy). The number of explanations can be decided by users of the system.

In a second exemplary embodiment, the present invention can be applied for generating synthetic patient samples with the specified health status and cohort characteristics. Here, a use case is to provide samples of artificially generated patients that will look like real, existing patients picked from a cohort of interest with predefined disease conditions. This would allow doctors to have a prototype of healthy and diseased patients for comparison to make more accurate and detailed analysis about microbiome patterns conditioned by geographical origin, habitat, or lifestyle. In this use case, the data source includes patient microbiome data of the cohort of interest and other cohorts. Application of the method according to an embodiment of the present invention provides to pick the patients with the desired characteristics, learn latent representations of the patients along with the transformations described in the method, and use the conditional VAE model generate synthetic data for the similar pseudo-patients in the target cohort space, as well as in any other cohort of interest. The generation of synthetic data also allows to preserve privacy of real patients. The output can be a synthetic patient with microbiome features, conditioned by the desired characteristics. Advantageously, any number of pseudo-patients of any hospital can be generated as explanations. Automated actions (technicity) can include generating effective treatments for diseases given characteristics derived from the synthetic data while maintaining privacy of real patients or without the need to obtain real microbiome data from patients.

In a third exemplary embodiment, the present invention can be applied to translate cohort-dependent patient characteristics across cohorts. Here, a use case is to transfer samples of real patients picked from one cohort into another cohort of interest. This allows doctors to discover how cohort-dependent patient characteristics change microbiome patterns of healthy or diseased patients depending on a cohort. This can also be used to find patients in other cohorts that are similar to the selected patient in the target cohort. In this use case, the data source includes patient microbiome data in the cohort of interest and other cohorts. Application of the method according to an embodiment of the present invention provides to transform a selected patient with the desired characteristics into the latent space using the encoder part of a VAE learned for the selected patient's original cohort and to generate data of that patient using the decoder part of a corresponding VAE learned for another cohort of interest. An automated action (technicity) can include determining a real patient with microbiome features translated into any cohort of interest. This can help in lab examination and experimentations to construct a vaccines or treatments which are effective for the cohort of interest.

For any of the exemplary embodiments, automated actions or technicity, in addition to providing the predictions and explanations, can include generation of a diagnosis or treatment plan, or administration of a drug or treatment.

In an embodiment, the present invention provides a method for interpretable domain adaptation for disease prediction across multiple cohorts, the method comprising the steps of:
1) Map patients of other cohorts to the space of the target cohort satisfying the constraints on the proximity of samples in source cohorts and the proximity about disease conditions (Step 2.1). The methods described herein can be used in any case of classification problem (e.g., binary, multi-label classification). The predictions are independent from each other and the system can predict multiple diseases simultaneously.
2) Learn patient distributions of different cohorts (Step 2.2). Patients can be grouped based on diseases and then proximity to each other can be determined from among the groups.
3) Correct patient distributions of source cohorts for the target cohort (Step 2.3).
4) Learn the disease model of the target cohort with the corrected multi-cohort data (Step 2.4).
5) Predict a disease for a new patient with the learned disease model (Step 2.5).
6) Select genuine patients that are similar with the new patient in the latent space or sample synthetic similar patients as explanations (Step 2.6).

Embodiments of the present invention provide for the following improvements and technical advantages over existing technology:
1) Learning a more precise disease model for the target cohort by augmenting patient microbiome data of the target cohort with the corrected patient microbiome data of multiple source cohorts, employing a method that:
   a. corrects the discrepancy in cross-cohort microbiome data caused by cross-cohort differences (e.g. different measurement techniques); and
   b. corrects the discrepancy in cross-cohort microbiome data caused by interpersonal differences (e.g. location, lifestyle etc.).
2) Mapping patient microbiome data of the source cohorts to the target cohort space with new constraints to better preserve semantic consistency of the microbiome data across cohorts and disease conditions, which includes:
   a. keeping proximity of the patients in the source cohort: where two patients of a source cohort are close to each other, then they are still similar after mapping to target cohort; and
   b. keeping proximity of the patients about disease conditions: where two patients from different cohorts have the same disease, then they should be more similar than those having different disease conditions.
3) Correcting misalignment of cross-cohort patient microbiome data, which uses generative neural networks to effectively approximate the distributions of the microbiome data in the target and source cohorts, and use the learned distributions to identify well-aligned patients from the source cohorts to facilitate the disease modeling in the target cohort. Compared to existing technology, this improves the accuracy of approximation of the microbiome data distributions and works well for the technically challenging small data size.

Embodiments of the present invention thus overcome a number of technical challenges and provide for a number of improvements to existing technology. Prior approaches suggest two main strategies to address the issue of generalizable disease predictions in cross-cohort microbiome studies: improving leveraging scheme of cross-cohort datasets and advancing machine learning methods.

Data leveraging approaches involve combining data samples from multiple cohorts to create a training dataset. Pasolli et al. propose adding healthy control samples from multiple studies, even if the diseases differ from the disease of interest. Song et al. consider incorporating part of the target cohort samples to a larger source cohort with the same disease of interest to generate the training dataset. However, these approaches are primarily based on empirical observations and do not account for the statistical properties of the cohorts, leaving the potential biases unexplained. In contrast, embodiments of the present invention introduce an interpretable domain adaptation method to predict disease and the corresponding explanations with multi-cohort patient data. The method according to embodiments of the present invention learns to correct the patient distributions of different cohorts, such that an accurate disease model of the target cohort can be learned from the corrected source cohorts.

From the machine learning perspective, few methods have been developed to address confounding factors in experimental design by explicitly incorporating meta-variables into the model, which may also help correct cohort-related biases. For instance, recent technology, referred to as Meta-Spec, encodes and embeds refined host variables (e.g., physiological characteristics, geography, and lifestyle habits of the patients) along with the sequenced microbiome features to make the predictions (see Wu, S., Li, Z., Chen, Y., Zhang, M., et al., “Deep learning and host variable embedding augment microbiome-based simultaneous detection of multiple diseases,” bioRxiv 2023.05.16.541058 (2023), which is hereby incorporated by reference herein). However, Meta-Spec assumes the availability and usage of patient-specific metadata, which can be challenging due to privacy concerns. In contrast, embodiments of the present invention provide to align data samples across cohorts and allows any high-performance method to be trained on top. In addition, the obtained patient distributions can be used to find similar genuine patients or sample pseudo-patients as explanations to overcome the technical challenge of the privacy of the data.

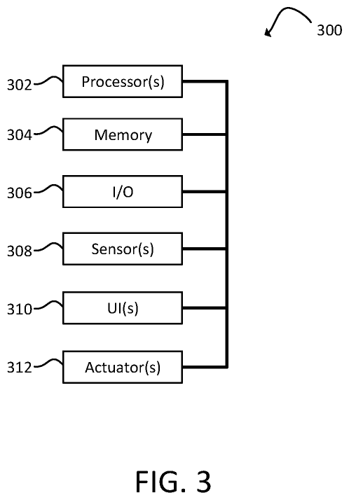

Referring to FIG. 3, a processing system 300 can include one or more processors 302, memory 304, one or more input/output devices 306, one or more sensors 308, one or more user interfaces 310, and one or more actuators 312. Processing system 300 can be representative of each computing system disclosed herein.

Processors 302 can include one or more distinct processors, each having one or more cores. Each of the distinct processors can have the same or different structure. Processors 302 can include one or more central processing units (CPUs), one or more graphics processing units (GPUs), circuitry (e.g., application specific integrated circuits (ASICs)), digital signal processors (DSPs), and the like. Processors 302 can be mounted to a common substrate or to multiple different substrates.

Processors 302 are configured to perform a certain function, method, or operation (e.g., are configured to provide for performance of a function, method, or operation) at least when one of the one or more of the distinct processors is capable of performing operations embodying the function, method, or operation. Processors 302 can perform operations embodying the function, method, or operation by, for example, executing code (e.g., interpreting scripts) stored on memory 304 and/or trafficking data through one or more ASICs. Processors 302, and thus processing system 300, can be configured to perform, automatically, any and all functions, methods, and operations disclosed herein. Therefore, processing system 300 can be configured to implement any of (e.g., all of) the protocols, devices, mechanisms, systems, and methods described herein.

For example, when the present invention states that a method or device performs task “X” (or that task “X” is performed), such a statement should be understood to disclose that processing system 300 can be configured to perform task “X”. Processing system 300 is configured to perform a function, method, or operation at least when processors 302 are configured to do the same.

Memory 304 can include volatile memory, non-volatile memory, and any other medium capable of storing data. Each of the volatile memory, non-volatile memory, and any other type of memory can include multiple different memory devices, located at multiple distinct locations and each having a different structure. Memory 304 can include remotely hosted (e.g., cloud) storage.

Examples of memory 304 include a non-transitory computer-readable media such as RAM, ROM, flash memory, EEPROM, any kind of optical storage disk such as a DVD, a Blu-Ray R disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 304.

Input-output devices 306 can include any component for trafficking data such as ports, antennas (i.e., transceivers), printed conductive paths, and the like. Input-output devices 306 can enable wired communication via USB®, Display Port®, HDMI®, Ethernet, and the like. Input-output devices 306 can enable electronic, optical, magnetic, and holographic, communication with suitable memory 306. Input-output devices 306 can enable wireless communication via WiFi®, Bluetooth®, cellular (e.g., LTE®, CDMA®, GSM®, WiMax®, NFC®), GPS, and the like. Input-output devices 306 can include wired and/or wireless communication pathways.

Sensors 308 can capture physical measurements of environment and report the same to processors 302. User interface 310 can include displays, physical buttons, speakers, microphones, keyboards, and the like. Actuators 312 can enable processors 302 to control mechanical forces.

Processing system 300 can be distributed. For example, some components of processing system 300 can reside in a remote hosted network service (e.g., a cloud computing environment) while other components of processing system 300 can reside in a local computing system. Processing system 300 can have a modular design where certain modules include a plurality of the features/functions shown in FIG. 3. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method for interpretable domain adaptation for cross-cohort predictions from medical data, the computer-implemented method comprising:
mapping patients of one or more source cohorts to a feature space of a target cohort based on constraints;
learning patient distributions of the one or more source cohorts and the target cohort; and
correcting the patient distributions of the one or more source cohorts for the target cohort.

2: The computer-implemented method according to claim 1, wherein the constraints include a proximity of samples in the one or more source cohorts, and the proximity about disease conditions.

3: The computer-implemented method according to claim 2, wherein mapping the patients of the one or more source cohorts to the feature space of the target cohort includes training an encoder model to embed the patients of the one or more source cohorts to the feature space of the target cohort, and wherein the constraints are utilized in a loss function as separate regularization terms while training the encoder model.

4: The computer-implemented method according to claim 1, wherein learning the patient distributions of the one or more source cohorts includes training a variational autoencoder to determine specific distributions for the target cohort and the one or more source cohorts.

5: The computer-implemented method according to claim 1, wherein correcting the patient distributions of the one or more source cohorts for the target cohort is based on generative deep learning and determining an alignment rate.

6: The computer-implemented method according to claim 1, further comprising learning a disease model of the target cohort using the one or more source cohorts that have corrected the patient distributions, the learned disease model being usable for obtaining an optimized prediction and/or supporting decision making.

7: The computer-implemented method according to claim 6, further comprising predicting a disease for a new patient based on the learned disease model.

8: The computer-implemented method according to claim 6, wherein the learned disease model revises loss by weighting the patients of the one or more source cohorts with an associated alignment rate.

9: The computer-implemented method according to claim 6, wherein the learned disease model generates one or more explanations for predicting the disease for the new patient.

10: The computer-implemented method according to claim 9, wherein the one or more explanations are based on genuine patients and/or synthetic patients used to obtain the disease prediction.

11: The computer-implemented method according to claim 1, wherein learning the patient distributions of the one or more source cohorts includes using the trained variational autoencoder or conditional variational autoencoder to learn the patient distributions separately, including pₜ(z|x) and pₜ(x|z) for the target cohort and pₛ(z|x) and pₛ(x|z) for each cohort of the one or more source cohorts.

12: The computer-implemented method according to claim 1, wherein the medical data includes microbiome data, wherein the alignment rate is a coefficient δ that specifies similarity of a patient i of a source cohort s of the one or more source cohorts is to the target cohort t, wherein correcting the patient distributions of the one or more source cohorts for the target cohort includes analyzing the patients of the source cohort s and the target cohort t by:
$${δ}_{t} ( {x}_{i}^{( s )} ) = \frac{{p}_{t} ( {x}_{i}^{( s )} )}{{p}_{s} ( {x}_{i}^{( s )} )} ,$$
wherein xᵢ^{(s)} denotes the microbiome data of the patient i of the source cohort s, wherein probability pₜ(xᵢ^{(s)}) is determined by:
$${p}_{t} ( {x}_{i}^{( s )} ) = \int {p}_{t} ( {z}_{i} ) {p}_{t} ( {x}_{i}^{( s )} \vert {z}_{i} ) {dz}_{i} \approx {\sum }_{j = 1}^{N} {p}_{t} ( {z}_{j} ) {p}_{t} ( {x}_{i}^{( s )} \vert {z}_{j} ) ,$$
wherein the term pₜ(xᵢ^{(s)}|zⱼ) is determined using the learned patient distributions, wherein pₜ(z) is determined by randomly drawing K patients from the target cohort and determining pₜ(z|xₖ^{(t)}) for each patient of the randomly drawn K patients, and wherein a mean distribution of the K patients is an empirical estimation of pₜ(z|x).

13: The computer-implemented method according to claim 1, wherein the medical data includes microbiome data, wherein learning the disease model includes using a neural network that generates disease labels as outputs and the microbiome data xᵢ^{(s)} of the corrected patient distributions of a corresponding source cohort of the one or more source cohorts and the microbiome data xⱼ^{(t)} of the corrected patient distributions of the target cohort as inputs, wherein parameters of the neural network are represented by Φ, wherein revising the loss includes:
$$Loss = {\sum }_{j = 1}^{M} - log p ( {y}_{j}^{( t )} \vert {x}_{j}^{( t )} , Φ ) + {\sum }_{s = 1}^{S} {\sum }_{i = 1}^{N} - log {δ}_{t} ( {x}_{i}^{( s )} ) p ( {y}_{i}^{( s )} \vert {x}_{i}^{( s )} , Φ ) ,$$
wherein p(yⱼ^{(t)}|xⱼ^{(t)}, Φ) represents predictions with data from the target cohort, and wherein p(yᵢ^{(s)}|xᵢ^{(s)}, Φ) represents the predictions with the data from the one or more source cohorts.

14: A computer system for interpretable domain adaptation for cross-cohort predictions from medical data, the computer system comprising one or more hardware processors which, alone or in combination, are configured to provide for execution of a machine learning method comprising the following steps:
mapping patients of one or more source cohorts to a feature space of a target cohort based on constraints;
learning patient distributions of the one or more source cohorts; and
correcting the patient distributions of the one or more source cohorts for the target cohort.

15: A tangible, non-transitory computer-readable medium having instructions thereon which, upon being executed by one or more processors, provide for interpretable domain adaptation for cross-cohort predictions from medical data by execution of a machine learning method comprising the following steps:
mapping patients of one or more source cohorts to a feature space of a target cohort based on constraints;
learning patient distributions of the one or more source cohorts; and
correcting the patient distributions of the one or more source cohorts for the target cohort.
